(19) United States
(12) Patent Application Publication
(10) Pub. No.: US 2022/0400984 A1
(43) Pub. Date: Dec. 22, 2022

(54) BIOSENSOR

(71) Applicant: DONGWOO FINE-CHEM CO., LTD., Iksan-si, Jeollabuk-do (KR)

(72) Inventors: Dong Yeop LEE, Pyeongtaek-si, Gyeonggi-do (KR); Dong Hee MA, Pyeongtaek-si, Gyeonggi-do (KR)

(73) Assignee: DONGWOO FINE-CHEM CO., LTD., Iksan-si, Jeollabuk-do (KR)

(21) Appl. No.: 17/770,906

(22) PCT Filed: Oct. 22, 2020
(86) PCT No.: PCT/KR2020/014498

§ 371 Date: Apr. 21, 2022

(30) Foreign Application Priority Data
Oct. 23, 2019 (KR) 10-2019-0132032

(51) Int. Cl. A61B 5/1486; A61B 5/145

(52) U.S. Cl. CPC A61B 5/1486; A61B 5/14546

(57) ABSTRACT

A biosensor includes a substrate; a working electrode including a working electrode layer formed on the substrate and an enzyme reaction layer formed on the working electrode layer to cover the working electrode layer; a reference electrode formed on the substrate to be spaced apart from the working electrode; and an insulation barrier rib separating the working electrode and the reference electrode on the substrate. The biosensor has a wide measurement range, excellent sensitivity, and reduced dispersion of measured values.

### TECHNICAL FIELD

The present invention relates to a biosensor. Particularly, the present invention relates to a biosensor having a wide measurement range, excellent sensitivity, and reduced dispersion of measured values.

### BACKGROUND ART

As the life expectancy of humans increases, the health care industry is expanding rapidly. In particular, there is an increasing demand for a portable and small biosensor that can conveniently measure various vital signs anywhere.

Biosensors use enzymes that react with chemical species contained in body fluids. When the enzyme reacts with the chemical species to generate an electric current, it is measured to measure the concentration of the chemical species [refer to Korean Patent Registration No. 10-0824731].

In a biosensor, selectivity, measurement range, reproducibility, response time, and lifetime are used as important indicators to judge the performance of the biosensor. In particular, since substances in a living body exist at different concentrations and the same substances may exist at different concentrations depending on the secreting organs, it is necessary to fabricate a sensor having a measurement range suitable for each purpose.

For example, lactate in vivo is present at a level of 2 to 10 mM in blood, but may be secreted at an average of 20 mM or more, and up to 50 mM or more in sweat. In order to detect a substance present in a high concentration in a living body as described above, it is essential to fabricate a sensor capable of measuring a wide measurement range, particularly a high concentration.

The conventional biosensor is manufactured by forming a working electrode layer and a reference electrode on a substrate, and dropping a composition for forming an enzyme reaction layer on the working electrode layer to cover the working electrode layer to form an enzyme reaction layer. In this case, in the process of forming the enzyme reaction layer, the area of the enzyme reaction layer participating in the reaction with the chemical species is reduced as the enzyme reaction layer is applied to the area beyond the working electrode layer area, thereby reducing the measurement range and sensitivity.

Therefore, there is a need for the development of a biosensor having a wide measurement range and excellent sensitivity.

In addition, in order to obtain a reliable evaluation result when measuring current through the biosensor, it is necessary to reduce the dispersion of measured values.

### DISCLOSURE OF INVENTION

### Technical Problem

An object of the present invention is to provide a biosensor having a wide measurement range, excellent sensitivity, and reduced dispersion of measured values.

### Technical Solution

According to an aspect, the present invention provides a biosensor comprising a substrate; a working electrode including a working electrode layer formed on the substrate and an enzyme reaction layer formed on the working electrode layer to cover the working electrode layer; a reference electrode formed on the substrate to be spaced apart from the working electrode; and an insulation barrier rib separating the working electrode and the reference electrode on the substrate.

In an embodiment of the present invention, the insulation barrier rib may define regions of the working electrode and the reference electrode.

In an embodiment of the present invention, a height of the insulation barrier rib may be higher than heights of the working electrode and the reference electrode.

In an embodiment of the present invention, a surface area ratio of the working electrode layer and the enzyme reaction layer may be 1:1.1 to 1:2.1.

In an embodiment of the present invention, the surface area ratio of the working electrode layer and the enzyme reaction layer may be 1:1.1 to 1:1.8.

In an embodiment of the present invention, the biosensor may be manufactured by forming the working electrode layer and the reference electrode on the substrate at a predetermined interval, forming the insulation barrier rib separating the working electrode layer and the reference electrode, and forming the enzyme reaction layer on the working electrode layer to cover the working electrode layer.

The biosensor according to an embodiment of the present invention may be used to measure a concentration of lactic acid, glucose, cholesterol, ascorbic acid, alcohol, or glutamic acid.

The biosensor according to an embodiment of the present invention may be used to measure the concentration of lactic acid.

### Advantageous Effects

The biosensor according to the present invention has an insulation barrier rib that separates the working electrode and the reference electrode, so that the enzyme reaction layer is not applied to an area that is too far out of the working electrode layer area, thereby preventing the amount of enzyme directly participating in the reaction from being reduced and increasing the measurement range of the biosensor. Accordingly, it is possible to detect chemical species present at a high concentration in the sensing target material, and the sensitivity can be improved. In addition, the biosensor according to the present invention can reduce the dispersion of measured values.

### DESCRIPTION OF DRAWINGS

FIG. 1 is a schematic cross-sectional view of a biosensor according to an embodiment of the present invention.

FIG. 2 shows the results of lactic acid measurement using the biosensor manufactured in Comparative Example 1.

FIG. 3 shows the results of lactic acid measurement using the biosensor manufactured in Comparative Example 2.

FIG. 4 shows the results of lactic acid measurement using the biosensor manufactured in Example 1.

FIG. 5 shows the results of lactic acid measurement using the biosensor manufactured in Example 2.

FIG. 6 shows the relative standard deviation of lactic acid measurement results using the biosensors manufactured in Comparative Examples 1 and 2 and Examples 1 and 2.

### BEST MODE

Hereinafter, the present invention will be described in detail with reference to the accompanying drawings.

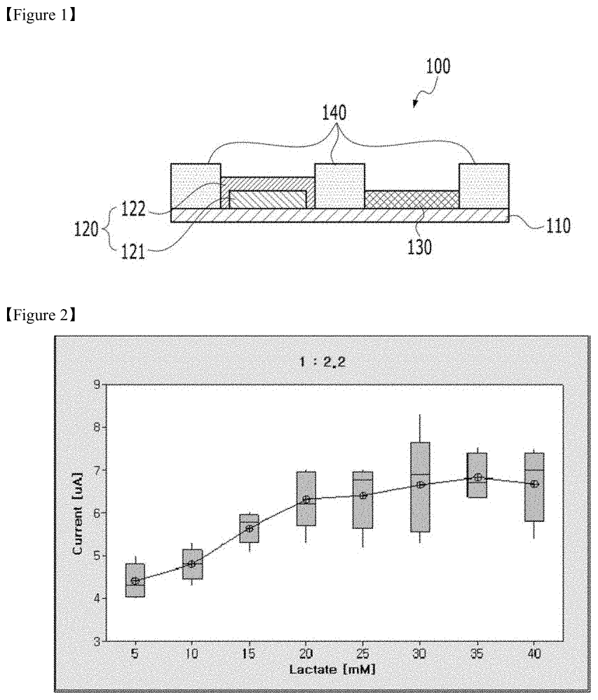

An embodiment of the present invention relates to a biosensor having an insulation barrier rib that separates a working electrode and a reference electrode. FIG. 1 is a schematic cross-sectional view of a biosensor according to an embodiment of the present invention.

Referring to FIG. 1, a biosensor 100 according to an embodiment of the present invention includes a substrate 110, a working electrode 120, a reference electrode 130, and an insulation barrier rib 140.

The substrate 110 functions to provide a structural base of the components constituting the biosensor.

For example, the substrate 110 may be implemented in the form of a base film having flexible characteristics.

Examples of specific materials that can be applied to the base film for implementing the substrate 110 may include thermoplastic resins, e.g., polyester resins such as polyethylene terephthalate, polyethylene isophthalate, polyethylene naphthalate and polybutylene terephthalate; cellulose resins such as diacetylcellulose and triacetylcellulose; polycarbonate resins; acrylate resins such as polymethyl (meth)acrylate and polyethyl (meth)acrylate; styrene resins such as polystyrene and acrylonitrile-styrene copolymer; polyolefin resins such as polyethylene, polypropylene, polyolefin having a cyclic or norbornene structure, and ethylene-propylene copolymer; vinyl chloride resins; amide resins such as nylon and aromatic polyamide; imide resins; polyethersulfone resins; sulfone resins; polyether ether ketone resins; polyphenylene sulfide resins; vinyl alcohol resins; vinylidene chloride resins; vinyl butyral resins; allylate resins; and polyoxymethylene resins. Also, a blend of the thermoplastic resins may be used. In addition, thermally curable or UV curable resins such as (meth)acrylate, urethane, acrylic urethane, epoxy and silicon resins may be used.

The base film may contain at least one suitable additive. Examples of the additive may include an UV absorber, an antioxidant, a lubricant, a plasticizer, a releasing agent, a coloring-preventing agent, an anti-flame agent, a nucleating agent, an anti-static agent, a pigment and a colorant. The base film may comprise various functional layers including a hard-coating layer, an anti-reflective layer and a gas barrier layer on one surface or both surface thereof, but the functional layer is not limited thereto. That is, other functional layers may also be included depending on the desired use.

If necessary, the base film may be surface-treated. For example, the surface treatment may be carried out by drying method such as plasma, corona and primer treatment, or by chemical method such as alkali treatment including saponification.

The substrate 110 may have a suitable thickness. Typically, considering workability in terms of strength and handling, or thin layer property, the thickness of the substrate may range from 1 to 500 μm, preferably 1 to 300 μm, more preferably 5 to 200 μm.

The working electrode 120 may undergo oxidation-reduction reaction of the sensing target material. The working electrode 120 includes a working electrode layer 121 and an enzyme reaction layer 122 formed on the working electrode layer. The working electrode 120 may detect an electrical signal generated by the reaction between the enzyme of the enzyme reaction layer 122 and the sensing target material. The sensing target material may be human sweat, body fluid, blood, etc., but is not limited thereto.

The working electrode layer 121 may be disposed on the substrate 110. For example, the working electrode layer 121 may be in contact with the substrate 110. The working electrode layer 121 may serve as a path through which electrons or holes generated in an oxidation-reduction reaction of a sensing target material are transmitted.

In an embodiment of the present invention, the working electrode layer 121 may include a carbon electrode layer. The carbon electrode layer may be formed of carbon paste. The carbon electrode layer may stably transport electrons and/or holes generated in the enzyme reaction layer 122.

In an embodiment of the present invention, the working electrode layer 121 may be formed of a single-layer carbon electrode layer formed of carbon paste. By using the carbon paste as an electrode in the form of a single layer, a metal electrode may be omitted. Accordingly, the biosensor 100 can be thinned.

In an embodiment of the present invention, the working electrode layer 121 may include a metal electrode layer. The metal electrode layer may include gold (Au), silver (Ag), copper (Cu), aluminum (Al), platinum (Pt), palladium (Pd), chromium (Cr), titanium (Ti), tungsten (W), niobium (Nb), tantalum (Ta), vanadium (V), iron (Fe), manganese (Mn), nickel (Ni), zinc (Zn), tin (Sn), molybdenum (Mo), cobalt (Co) or an alloy thereof (e.g., silver-palladium-copper (APC)). These may be used alone or in combination of two or more. The metal electrode layer may be formed of at least one of Au, Ag, APC alloy, and Pt. The Au, Ag, APC alloy, and Pt may improve electrical conductivity of the working electrode layer 121 and reduce resistance. Accordingly, the detection performance of the biosensor 100 may be improved.

In an embodiment of the present invention, the working electrode layer 121 may include both the metal electrode layer and the carbon electrode layer described above. In this case, the metal electrode layer may be disposed on the bottom surface of the carbon electrode layer. The metal electrode layer may be in contact with the substrate 110. The carbon electrode layer may be in contact with the enzyme reaction layer 122.

When the working electrode layer 121 includes a metal electrode layer, a metal protective layer may be additionally formed on an upper surface and/or a bottom surface of the metal electrode layer. The metal protective layer may entirely cover the upper surface of the metal electrode layer while having electrical conductivity. For example, the metal protective layer may be in direct contact with the metal electrode layer. The metal protective layer may prevent oxidation-reduction of the metal electrode layer due to the oxidation-reduction reaction of the working electrode 120, thereby improving reliability of an electrical signal detected by the working electrode 120.

For example, the metal protective layer may include indium tin oxide (ITO), indium zinc oxide (IZO), or the like. For example, the metal protective layer may be formed of only ITO or IZO. The ITO and IZO are chemically stable while having electrical conductivity, so that the metal electrode layer can be effectively protected from the oxidation-reduction reaction.

The working electrode layer 121 may include an electron transport material.

The electron transport material may be, for example, a material that is oxidized or reduced by receiving electrons/holes generated in an oxidation-reduction reaction of the sensing target material in the enzyme reaction layer 122.

As the electron transport material, Prussian blue (Fe₄[Fe(CN)₆]₃), potassium ferricyanide (K₃[Fe(CN)₆]), potassium iron ferrocyanide (KFeIII[FeII(CN)₆]·xH₂O), ferrocene, ruthenium, etc. can be used, and it is possible to apply it in a separate process or by integrating it with carbon paste.

Prussian blue is a blue pigment and may have high oxidation properties. When Prussian blue is used for the working electrode layer 121 as an electron transport material, the electrical sensitivity of the working electrode 120 may be improved.

The electron transport material may be included in an amount of 0.05 to 1 wt % based on 100 wt % of the working electrode layer 121. When the content of the electron transport material satisfies the above range, the sensitivity range (e.g., upper limit) for the sensing target material may be increased. When the content of the electron transport material is less than the above range, the sensing range of the sensing target material may be reduced. When the content of the electron transport material exceeds the above range, the electron transport material aggregates with each other, thereby reducing sensing performance. Preferably, the electron transport material may be included in an amount of 0.1 to 0.5 wt % based on 100 wt % of the working electrode layer 121.

The working electrode layer 121 may be formed by printing carbon paste on the substrate 110 or by forming a metal film and then patterning it.

For the patterning, a patterning method commonly used in the art may be used. For example, photolithography may be used.

When the working electrode layer 121 further includes a metal protective layer, the metal electrode layer may be first patterned and then the metal protective layer may be formed. Alternatively, after an ITO or IZO conductive oxide film is formed on the metal film, the metal film and the conductive oxide film may be patterned together to form a metal electrode layer and a metal protective layer together.

The enzyme reaction layer 122 may be disposed on the working electrode layer 121. The enzyme reaction layer 122 may be formed on the working electrode layer to cover the working electrode layer. The enzyme reaction layer 122 is provided as a layer in which a chemical reaction of the sensing target material occurs.

The enzyme reaction layer 122 may include an oxidase or a dehydrogenase. The oxidase and the dehydrogenase may be selected according to the type of the sensing target material.

The oxidase may include at least one of lactate oxidase, glucose oxidase, cholesterol oxidase, ascorbic acid oxidase, and alcohol oxidase.

The dehydrogenase may include at least one of lactate dehydrogenase, glucose dehydrogenase, glutamate dehydrogenase, and alcohol dehydrogenase.

Therefore, the biosensor according to the present invention can be used to measure the concentration of lactic acid, glucose, cholesterol, ascorbic acid, alcohol or glutamic acid, particularly lactic acid.

The enzyme reaction layer 122 may further include a mediator. Examples of the mediator include potassium ferricyanide, cytochrome C, pyrroroquinolinequinone (PQQ), NAD⁺, NADP⁺, a copper complex, a ruthenium compound, phenazinemethosulphate and derivatives thereof, and these may be used alone, or in combination of two or more.

In addition, the enzyme reaction layer 122 may additionally include a water-soluble polymer such as modified polyvinyl alcohol or polyvinylpyrrolidone, which is a high molecular material, in order to act as a filter or improve stability when a high-concentration sensing target material is absorbed.

When a sample including a sensing target material is injected into the biosensor 100, the sensing target material included in the sample may react with an oxidase or a dehydrogenase to generate a by-product such as hydrogen peroxide. At this time, the electron transport material (e.g., Prussian blue) may reduce the by-product, and may itself be oxidized. The oxidized electron transport material may be reduced again by obtaining electrons from the electrode surface to which a predetermined voltage is applied.

The concentration of the sensing target material in the sample is proportional to the amount of current generated during the oxidation of the electron transport material. Accordingly, the concentration of the sensing target material may be measured by measuring the amount of current.

The oxidase or dehydrogenase may be immobilized through a binder. The binder may include a binder commonly used in the art, for example, an organic material or an inorganic material such as Nafion or a derivative thereof, chitosan, bovine serum albumin (BSA), or Si gel.

It is also possible to add a small amount of acid or base to the enzyme reaction layer 122 to adjust pH or increase solubility.

In an embodiment of the present invention, the ratio of the surface area of the working electrode layer to the enzyme reaction layer may be 1:1.1 to 1:2.1, preferably 1:1.1 to 1:1.8. If the surface area ratio of the working electrode layer to the enzyme reaction layer is less than 1:1.1, the dispersion of the measured result increases, the electrode is damaged due to exposure, and there may be difficulties in the manufacturing process. If it exceeds 1:2.1, the amount of enzymes that can directly participate in the reaction may decrease, which may reduce sensitivity and measurement range.

The surface area ratio of the working electrode layer and the enzyme reaction layer can be adjusted by controlling the surface area of the working electrode layer formed in a region limited by the insulation barrier rib, and controlling the flow of the composition for forming the enzyme reaction layer by setting the height of the insulation barrier rib to be higher than the height of the working electrode layer and the enzyme reaction layer.

The ratio of the surface area between the working electrode layer and the enzyme reaction layer can be controlled by applying the composition for forming the enzyme reaction layer by artificially adjusting the application range while applying the same amount.

The surface area of the working electrode layer and the enzyme reaction layer means the surface area of the upper surface.

A protective layer (not shown) may be additionally formed on the upper surface of the enzyme reaction layer 122.

The protective layer may protect the enzyme reaction layer 122 from external impact and chemical substances other than the sensing target material.

The protective layer can pass only the sensing target material. Accordingly, it is possible to prevent the enzyme reaction layer 122 from being denatured or damaged by a material other than the sensing target material.

As the protective layer, an ion exchange membrane commonly used in the art may be used as long as the sensing target material passes therethrough.

The ion exchange membrane may include a cation exchange resin such as a perfluorosulfonic acid resin. For example, the ion exchange membrane may include Nafion, etc. as a commercially available product, but this is only an example, and it is not limited thereto.

The total thickness of the enzyme reaction layer and the protective layer may be 1 to 10 μm, preferably 2 to 5 μm. If the total thickness of the enzyme reaction layer and the protective layer is less than 1 μm, the current may decrease or it may be difficult to sufficiently exert the role of the protective layer. If it exceeds 10 μm, the reaction rate may be reduced.

The enzyme reaction layer 122 may be formed by applying a composition obtained by mixing an oxidase or a dehydrogenase with a binder on the working electrode layer 121 followed by drying.

The reference electrode 130 may be disposed on the substrate 110. The reference electrode 130 may be disposed on the same surface of the substrate 110 on which the working electrode 120 is disposed. The reference electrode 130 may be disposed to be spaced apart from the working electrode 120. The reference electrode 130 and the working electrode 120 may be electrically disconnected.

The reference electrode 130 may provide a reference value for a current value or a potential value measured by the working electrode 120 during measurement. By using the potential value of the reference electrode 130 as a reference value, the oxidation-reduction reaction of the sensing target material occurring in the working electrode 120 may be specified.

In addition, by comparing the reference value of the current value with the current value measured by the working electrode 120, it is possible to calculate the amount of current changed purely by the measurement target component (e.g., the sensing target material), and the concentration of the measurement target component can be derived from the current amount.

The reference electrode 130 may include, for example, an Ag/AgCl electrode layer. The Ag/AgCl electrode layer may be formed from Ag/AgCl paste.

Since the reference electrode 130 may be damaged when an overcurrent flows, it is preferable to control the size of the surface area to be 0.7 to 1.3 compared to the area of the working electrode.

The surface area of the reference electrode 130 can be appropriately controlled by adjusting the size of the region defined by the insulation barrier rib when the insulation barrier rib is formed on the substrate.

The insulation barrier rib 140 separates the working electrode and the reference electrode to control the application range of the enzyme reaction layer. The insulation barrier rib 140 may define regions of the working electrode and the reference electrode. Accordingly, since the enzyme reaction layer is not applied to the area too far beyond the working electrode layer area, the amount of the enzyme directly participating in the reaction is prevented from decreasing, thereby increasing the measurement range of the biosensor and improving the sensitivity. In addition, the insulation barrier rib 140 may control the size of the exposed reference electrode 130, and may serve as a barrier to an external interference material.

As the insulation barrier rib 140, any insulation material can be used without limitation, but preferably, an oxide-based or nitride-based inorganic insulation material, or a UV curing type using a photoinitiator or thermosetting type organic polymer material may be used. Specifically, as the material of the insulation barrier rib 140, silicon oxide, acrylic resin, polyester, polyimide, polytetrafluoroethene (PTFE), poly(p-xylylene), or the like may be used, and these may be used alone or in combination of two or more.

The insulation barrier rib 140 may be formed by screen-printing, photolithography, sputtering, or chemical vapor deposition (CVD).

A height of the insulation barrier rib may be higher than heights of the working electrode and the reference electrode.

The height of the insulation barrier rib 140 may be 1 to 40 μm, and preferably 5 to 30 μm in consideration of the amount of the enzyme reaction layer material applied. When the height of the insulation barrier rib 140 is less than 1 μm, the composition for forming the enzyme reaction layer may invade onto the insulation barrier rib when the enzyme reaction layer is manufactured, or even the reference electrode may be severely invaded. If it exceeds 40 μm, it may be difficult to apply the measurement substrate or the drying time of the insulation barrier rib may get longer.

Although not shown in the drawings, each of the working electrode 120 and the reference electrode 130 is connected to wiring. The wiring connected to the working electrode 120 and the wiring connected to the reference electrode 130 may be electrically spaced apart from each other. The wiring may be connected to a driver integrated circuit (IC) chip.

The wiring may be formed of the same material as the working electrode layer 121 of the working electrode 120, and may be formed of the same material as the reference electrode 130.

The wiring may be integrally formed with the working electrode layer 121 and the reference electrode 130. The wiring may be integrally formed by forming a carbon paste film and/or a metal film on the substrate 110 and patterning it. Alternatively, the working electrode layer 121, the reference electrode 130, and the wiring may be integrally formed through a screen-printing method.

Electrical signals measured from the working electrode 120 and the reference electrode 130 may be transmitted to the driver IC chip through the wiring, and the driver IC chip may calculate the concentration of the measurement target component.

The biosensor according to an embodiment of the present invention may be prepared by forming a working electrode layer and a reference electrode on the substrate at a predetermined interval, forming an insulation barrier rib separating the working electrode layer and the reference electrode, and then forming an enzyme reaction layer on the working electrode layer to cover the working electrode layer.

In particular, the biosensor 100 according to the present invention can be used to measure lactate (lactic acid). For example, as the intensity and duration of exercise increases during exercise, lactic acid level in the body may increase. The lactic acid may be excreted outside the body through sweat, and the concentration of lactic acid discharged may be measured through the biosensor 100. The biosensor 100 according to the present invention has the insulation barrier rib that separates the working electrode and the reference electrode, so that the enzyme reaction layer is not applied to an area that is too far out of the working electrode layer area, thereby preventing the amount of enzyme directly participating in the reaction from being reduced and increasing the measurement range of the biosensor. Accordingly, it is possible to detect chemical species present at a high concentration in the sensing target material, for example, lactic acid having a concentration of 50 mM or more in sweat, and the sensitivity can be improved.

The biosensor 100 according to the present invention can increase the sensitivity range by controlling the surface area ratio of the enzyme reaction layer and the working electrode layer, and can reduce the dispersion of measured values through uniform application.

In addition, the biosensor 100 according to the present invention can prevent the spreadability of the composition for forming an enzyme reaction layer that may occur during the manufacturing process, so that it can have an enzyme reaction layer formed uniformly.

The biosensor 100 according to the present invention may be manufactured in the form of a patch.

Hereinafter, the present invention will be described in more detail by way of Examples, Comparative Examples and Experimental Examples. These Examples, Comparative Examples, and Experimental Examples are only for illustrating the present invention, and it is apparent to those skilled in the art that the scope of the present invention is not limited thereto.

### Example 1: Fabrication of a Biosensor

A biosensor was fabricated in the same structure as the embodiment of FIG. 1.

A PET film having a thickness of 180 μm was used as a substrate.

A working electrode layer was formed by screen-printing carbon paste (DS-7406CB, manufactured by Daejoo Electronic Materials) on the substrate.

At a certain distance from the working electrode layer, Ag/AgCl (DBS-4585V, manufactured by Daejoo Electronic Materials) was screen-printed to form a reference electrode.

An insulation barrier rib separating the working electrode and the reference electrode was formed as follows.

Acrylic resin (DGMR-011, made by Daejoo Electronic Materials) was used as the insulation barrier rib, and it was formed at a position where the working electrode and the reference electrode could be separated by screen-printing, and was cured by irradiating UV.

An enzyme reaction layer was formed on the working electrode layer to prepare a working electrode.

The enzyme reaction layer was prepared as follows.

20 wt % of 0.0016% 1-methoxy-5-methylphenazinium methyl sulfate (manufactured by Sigma Aldrich) was added to 20 wt % of lactate oxidase 4 U/1 μl (manufactured by TOYOBO, 10 U/1 μl stock solution) and mixed evenly, and then 60 wt % of phosphate-buffered saline (PBS) was added and mixed evenly to prepare a composition for forming the enzyme reaction layer. 0.0016% 1-methoxy-5-methylphenazinium methyl sulfate was prepared by diluting it with PBS, and lactate oxidase was also prepared by diluting it with PBS. 2.0 μl of the above composition for forming the enzyme reaction layer was dropped onto the working electrode layer, and then dried at room temperature for about 30 minutes and under an N2 atmosphere for about 20 minutes to form the enzyme reaction layer.

The surface area ratio of the working electrode layer and the enzyme reaction layer was 1:1.5.

### Example 2: Fabrication of a Biosensor

A biosensor was fabricated in the same manner as in Example 1, except that the surface area ratio of the working electrode layer and the enzyme reaction layer was controlled to be 1:1.2.

### Comparative Example 1: Fabrication of a Biosensor

A biosensor was fabricated in the same manner as in Example 1, except that the insulation barrier rib was not formed.

The surface area ratio of the working electrode layer and the enzyme reaction layer was 1:2.2.

### Comparative Example 2: Fabrication of a Biosensor

A biosensor was fabricated in the same manner as in Example 1, except that the surface area ratio of the working electrode layer and the enzyme reaction layer was controlled to be 1:0.8.

### Experimental Example 1: Measurement of Lactic Acid

Lactic acid was measured as follows using the biosensors manufactured in Examples and Comparative Examples. As a sample, a lactic acid combination solution having a concentration of 5, 10, 15, 20, 25, 30, 35, 40 mM or more was used, and an electrochemical analysis equipment CHI630 (CH Instruments) was used as a measuring device.

Measurement was performed by supplying a sample to the biosensor and applying a voltage of 200 mV for 30 seconds after sensing the sample.

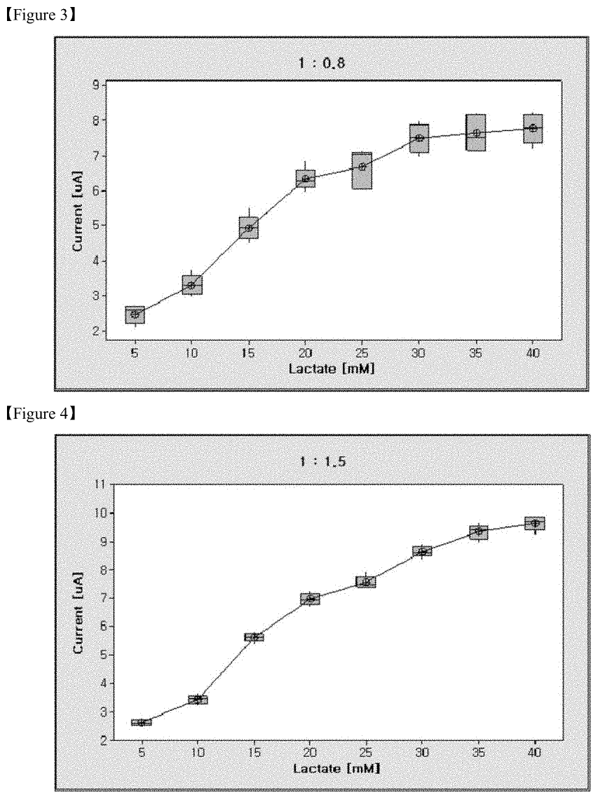

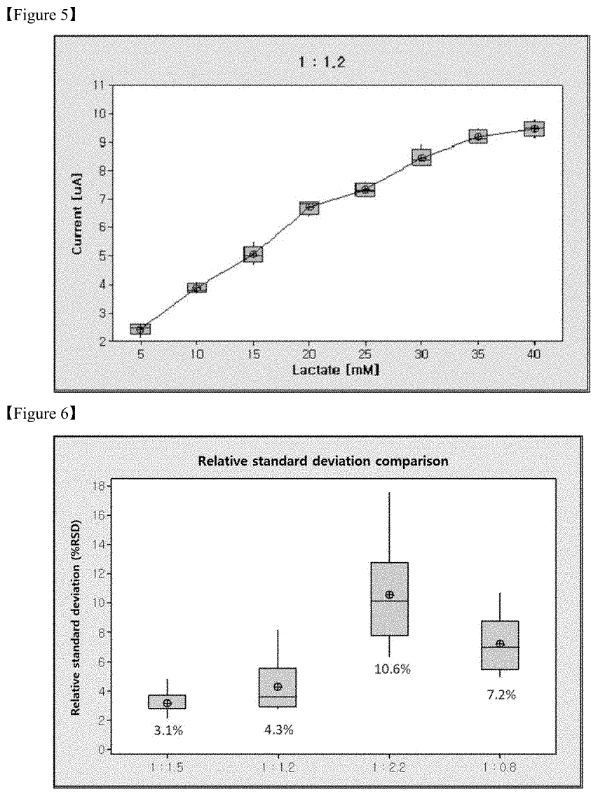

The measurement results of Comparative Examples 1 and 2 are shown in FIGS. 2 and 3, respectively, and the measurement results of Examples 1 and 2 are shown in FIGS. 4 and 5, respectively.

In order to compare the current value dispersion according to the presence or absence of the insulation barrier rib, the relative standard deviation (% RSD) of the measurement result was calculated by Equation 1 below and is shown in FIG. 6.

% RSD=standard deviation/mean×100 [Equation 1]

In addition, the measurement results are summarized in Table 1 below.

TABLE 1

| | | | Comparative | Comparative |
|---|---|---|---|---|
| | Example 1 | Example 2 | Example 1 | Example 2 |
| Surface area ratio | 1:1.5 | 1:1.2 | 1:2.2 | 1:0.8 |
| Measurement | 5 mM~40 mM | 5 mM~40 mM | 5 mM~20 mM | 5 mM~30 mM |
| range | | | | |
| Relative standard | 3.1% | 4.3% | 10.6% | 7.2% |
| deviation | | | | |

As shown in FIGS. 2 to 5, the biosensor of Comparative Example 1 without an insulation barrier rib had a maximum detectable lactic acid concentration of 20 mM, and the measured value reached saturation in a concentration range exceeding 20 mM, but the biosensors of Examples 1 and 2 were found to be capable of detecting up to a concentration of 40 mM. In addition, the biosensor of Comparative Example 2, in which the surface area ratio of the working electrode layer and the enzyme reaction layer was 1:0.8, showed that the maximum detectable lactic acid concentration was as low as 30 mM.

In addition, through Table 1 and FIGS. 2 to 6, it was confirmed that the biosensors of Comparative Examples 1 and 2 showed large dispersion of current values, but the biosensors of Examples 1 and 2 had small dispersion of current values.

Therefore, it was found that the dispersion of the measured values of the biosensor decreased as the insulation barrier rib was provided.

Although particular embodiments of the present invention have been shown and described, it will be understood by those skilled in the art that it is not intended to limit the present invention to the preferred embodiments, and it will be obvious to those skilled in the art that various changes and modifications may be made without departing from the spirit and scope of the invention.

The scope of the present invention, therefore, is to be defined by the appended claims and equivalents thereof.

1. A biosensor comprising:
a substrate;
a working electrode including a working electrode layer formed on the substrate and an enzyme reaction layer formed on the working electrode layer to cover the working electrode layer;
a reference electrode formed on the substrate to be spaced apart from the working electrode; and
an insulation barrier rib separating the working electrode and the reference electrode on the substrate.

2. The biosensor according to claim 1, wherein the insulation barrier rib defines regions of the working electrode and the reference electrode.

3. The biosensor according to claim 1, wherein a height of the insulation barrier rib is higher than heights of the working electrode and the reference electrode.

4. The biosensor according to claim 1, wherein a surface area ratio of the working electrode layer and the enzyme reaction layer is 1:1.1 to 1:2.1.

5. The biosensor according to claim 4, wherein the surface area ratio of the working electrode layer and the enzyme reaction layer is 1:1.1 to 1:1.8.

6. The biosensor according to claim 1, wherein the biosensor is manufactured by forming the working electrode layer and the reference electrode on the substrate at a predetermined interval, forming the insulation barrier rib separating the working electrode layer and the reference electrode, and forming the enzyme reaction layer on the working electrode layer to cover the working electrode layer.

7. The biosensor according to claim 1, wherein the biosensor is used to measure a concentration of lactic acid, glucose, cholesterol, ascorbic acid, alcohol, or glutamic acid.

8. The biosensor according to claim 7, wherein the biosensor is used to measure the concentration of lactic acid.
